(19) United States
(12) Patent Application Publication
(10) Pub. No.: US 2017/0319739 A1
(43) Pub. Date: Nov. 9, 2017

(54) CHEMICALLY FOAMING HEMOSTAT

(71) Applicant: Joshua R&D Technologies, LLC, Plano, TX (US)

(72) Inventor: David Pickett, Plano, TX (US)

(21) Appl. No.: 15/150,094

(22) Filed: May 9, 2016

(51) Int. Cl. A61L 24/00

(52) U.S. Cl. CPC A61L 24/0036; A61L 24/0015; A61L 2400/04

(57) ABSTRACT

A chemically foaming hemostat may use a liquid gel, a dried gel, a dry powder, or a solid “plug” like medium including a foaming agent to stop bleeding. The hemostat also may introduce pharmaceuticals and/or other compounds into a wound to control/mitigate shock, calm the patient, administer antibiotics, administer anti-sepsis compounds, control infection, control sepsis, and/or mitigate pain. No foreign matter need be introduced in connection with use of a chemically foaming hemostat. This eliminates the need to surgically remove introduced foreign matter at a later time. Use of a chemically foaming hemostat also may reduce the likelihood of trauma that sometimes occurs when foreign matter is left in place inside a wound.

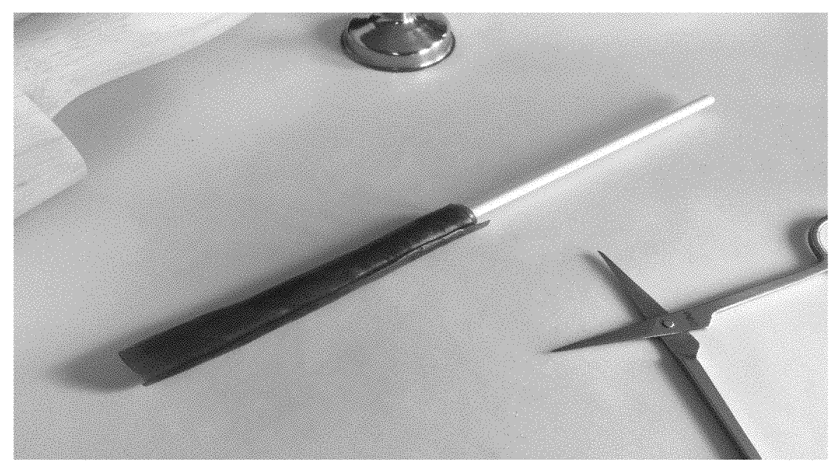

### FIELD OF THE DISCLOSURE

The present disclosure generally relates to hemostats, and more particularly, to a chemically foaming hemostat.

### BACKGROUND

A major cause of death from major accidents and war is blood loss from uncontrollable bleeding, such as may be suffered from a severe wound where the patient loses so much blood that his/her body can no longer function. Not all such blood loss is rapid; an unconscious, disabled, helpless, and/or unattended victim may even bleed to death from a relatively slow continuous, unabated flow. Internal soft tissue injuries that may not be outwardly visible to medical personnel also are a leading cause of death due to blood loss.

When there is excessive bleeding from puncture/tear wounds, tissues associated with the wound can be exposed to bacteria, viruses, mold, spores and fungi. This contamination—known as sepsis—can be controlled through use of antiseptics, such as alcohols, quaternary ammonium compounds, chlorhexidine gluconate, hydrogen peroxide, and nano silver, among others that may kill pathogens on contact. Tissues where pathogens are not killed can cause infections, requiring introduction of antibiotics. Uncontrolled infections can cause tissue death resulting in organ death and infection of multiple organs and systems, resulting in patient disabilities or death.

Presently, the primary means for a first responder or other medical personnel to stop blood loss is through the use of pressure, a mechanical hemostat instrument/tool, and/or a tourniquet and/or a field dressing or gauze pad pressed firmly against the wound to close/pinch off the artery or vein from which blood flows. These methodologies may absorb blood and encourage clotting until surgery can be performed. Unfortunately, at the same time, there also may be clotting of the bandages, dressings, sutures, packings, sponges, and other foreign matter into the wound, often requiring even more extensive surgery to remove these foreign materials from the wound and even more blood loss, stress and shock to the patient. Also, if the foreign matter is not completely removed from a wound, infections, cysts, and even death may occur.

Surgery can permanently stop the blood loss, but often there can be a long time period between the incident and surgery. A first responder or other medical personnel may have expertise at stopping bleeding, but still may not be able to stop massive bleeding, bleeding from numerous wounds or—more typically—wounds positioned such that application of pressure does not pinch off the “bleeders” and/or the injuries or wounds may be so profound that arteries or veins are not available to pinch off

### SUMMARY

Embodiments of the present disclosure may provide a chemically foaming hemostat comprising a foaming agent, such as a foaming gel or liquid or powder, formed from blending a gelling agent, a sterile anhydrous liquid, a foaming agent and a pharmaceutical-type clotting agent, selected pharmaceuticals including shock mitigation pharmaceuticals, sepsis mitigation, pain mitigation, infection mediation, other selected pharmaceuticals and compounds and an insertion mechanism affixed to the foaming gel or liquid or powder to facilitate introduction of the foaming gel or liquid or powder into a wound, wherein the foaming gel or liquid or powder may foam upon contact with blood in the wound and abate bleeding. The foaming gel or liquid or powder may further include one or more compounds selected from the group comprising: clotting agents, shock mitigation agents, antibiotics, nano-silver particles, pain relieving agents, and anti-sepsis agents, dyes and markers, wherein upon activation of the foaming gel or liquid or powder in the wound, the one or more compounds may be introduced into the wound. The foaming gel or liquid or powder may be applied to the insertion mechanism/stick/tube and dried. The foaming gel may be rolled into thin sheets for application to the insertion mechanism which may assume a shape similar to a popsicle, with the foaming gel and pharmaceuticals/medicines formed into a mass at the end of the insertion mechanism/stick/tube. The foaming gel may be poured into a mold for application to the insertion mechanism. The insertion mechanism may be a sterile stick or tube or wire and may further include a cover shield tube covering the sterile stick or wire, wherein upon insertion of the chemically foaming hemostat into the wound, the sterile stick may hold the foaming gel in pace and the optional cover shield tube may then be withdrawn. The chemically foaming hemostat may further include a radiation marker, radioactive markers, x-ray markers, one or more sensors, detectors or wires to monitor the wound, and/or one or more tubes to evacuate and introduce liquid into the wound.

Other embodiments of the present disclosure may provide a foaming gel or liquid or powder for use in a chemically foaming hemostat, the foaming gel or liquid or powder comprising a first mixture formed from mixing a gelling agent with a sterile anhydrous liquid. Then a foaming agent and a pharmaceutical-type clotting agent may be added to the first mixture and blended to form the foaming gel of the chemically foaming hemostat, wherein upon coming into contact with blood in a wound, the foaming gel or liquid or powder may foam and bleeding in the wound may abate due to the pressure of the foaming action and due to the chemicals and pharmaceuticals that may be present in the foam. The second mixture may further include one or more additional compounds selected from the group comprising: shock mitigation agents, antibiotics, nano-silver particles, pain relieving agents, anti-sepsis agents and other selected agents and pharmaceuticals that may be desirable to place into the wound. The foaming gel may be rolled into one or more thin sheets and applied/rolled onto to a sterile stick or tube or wire that may be used to introduce the foaming gel into the wound, wherein upon application, the foaming gel containing the sterile stick or tube or wire may be dried. The foaming gel may be formed into a solid shape and applied to a sterile stick or tube or wire that may be used to introduce the foaming gel into the wound, wherein upon application, the foaming gel containing the sterile stick or tube or wire may be dried. The foaming gel may be poured into a mold, preferably non-stick, a sterile stick or tube or wire may be inserted into the foaming gel, and the foaming gel containing the sterile stick may be dried. The foaming gel may remain in liquid form for introduction into the wound. The foaming gel may be placed into powder form or anhydrous liquid form for introduction into the wound. The foaming gel may be formed as sheets to be applied topically to the wound. The foaming gel may be formed as a powder or an anhydrous liquid to be applied topically to the wound.

Further embodiments of the present disclosure may provide a method for using a chemically foaming hemostat, the method comprising introducing the chemically foaming hemostat formed of a foaming agent affixed to a sterile stick or wire into a wound where blood is present, and removing the sterile stick or wire from the wound when the foaming agent has been introduced into the wound, wherein upon contact with the blood, the foaming agent may activate to foam in the wound and bleeding associated with the wound may stop due to the pressure of the foaming action or the action of the chemicals/pharmaceuticals that may be present in the foam. The foaming agent may be impregnated with one or more compounds selected from the group comprising: clotting agents, shock mitigation agents, antibiotics, nano-silver particles, pain relieving agents, and anti-sepsis agents, wherein upon activation of the chemically foaming hemostat in the wound, the one or more compounds may be introduced into the wound tissues. The foaming hemostat may be applied in a gel form, a powder form or in a liquid form.

Other technical features may be readily apparent to one skilled in the art from the following figures, descriptions and claims.

### BRIEF DESCRIPTION OF THE DRAWING

For a more complete understanding of this disclosure, reference is now made to the following description, taken in conjunction with the accompanying drawing, in which:

The FIGURE depicts a chemically foaming hemostat according to an embodiment of the present disclosure.

### DETAILED DESCRIPTION

Embodiments of the present disclosure relate to hemostasis and control of excessive bleeding even in the presence of contaminants, leading to wound healing. In hemostasis, a wound may be sealed until the tissues can be repaired. Using a chemically foaming hemostat according to embodiments of the present disclosure may stop bleeding while leaving no foreign matter and also may allow for introduction of pharmaceuticals and/or other compounds into the wound and/or place sensors in the wound to further treat and monitor a patient. A chemically foaming hemostat according to embodiments of the present disclosure may utilize a foaming gel or powder or liquid, whether taking the form of a liquid gel, a dried gel, a powder, or a solid, or a liquid that may foam when placed in contact with blood in the wound. This foaming action may pack the wound and stop bleeding associated with the wound in even the most grievous and deep wounds, at least until surgical treatment may be administered (which may sometimes be several hours). The foaming action in combination with the pressure associated with applying a wound dressing and/or manually applied pressure may cause the foam to increase its pressure within the wound that may then press the pharmaceuticals and other agents into the damaged tissues and arteries and veins where the agents will be in contact with the injured tissues. The foam pressure may help, or equal or exceed the blood pressure, further stopping the blow of blood and further pressing the chemical and pharmaceutical agents into the damaged tissues. The foaming gel or power or liquid also may be used as a carrier for one or more pharmaceuticals or other compounds that may distributed within the wound to create a barrier to block contaminants and also treat other conditions associated with the wound and damaged tissues and/or the patient himself/herself.

A chemically foaming hemostat according to embodiments of the present disclosure also may stop internal bleeding when the location of the injury can be reasonably approximated. An incision may be made in the location, and then the bleeding area may be packed with the chemically foaming hemostat. The patient may be stabilized long-term, and the triage level may be lowered. No foreign matter (i.e., bandages, sponges, dressings, and tapes) need be introduced in connection with use of a chemically foaming hemostat according to embodiments of the present disclosure. This eliminates the need to surgically remove foreign matter at a later time. It also reduces the likelihood of trauma that sometimes occurs when foreign matter is left in place inside a wound. The reduction of surgical time may prevent the patient from dying in surgery or afterward from shock and trauma and other conditions caused by long surgery procedures, including reduced loss of the patient's natural blood.

As described in more detail herein, a chemically foaming hemostat according to some embodiments of the present disclosure may operate in a manner similar to a tampon in that a plug formed of a foaming gel may be inserted into a wound using a sterile insertion stick. The plug in the chemically foaming hemostat may be impregnated with pharmaceuticals and/or other compounds that may be used to stop bleeding, control/mitigate shock, calm the patient, administer antibiotics, administer anti-sepsis compounds, control infection, control sepsis, and mitigate pain. However, other types of pharmaceuticals and/or compounds may be introduced to address other issues without departing from the present disclosure.

A chemically foaming hemostat according to embodiments of the present disclosure may include a foaming gel or liquid or powder, when activated by contact with blood, introduces clotting pharmaceuticals and/or other compounds and pharmaceuticals to the wound. The foaming gel of the chemically foaming hemostat may be uniformly applied directly to the wound and/or leaking blood vessels.

In an embodiment of the present disclosure, the foaming gel may be formed by mixing prescription-grade collagen powder (or a similar gelling agent) with a sterile anhydrous liquid, such as anhydrous ethyl alcohol or a light evaporative anhydrous oil that may dry/flash off in a freeze-dry process or in a low temperature oven. This mixing step may be performed under a sterile laminar flow air hood or another similar environment. Once mixed, the water/blood-activated foaming agent may then be added along with a pharmaceutical-type clotting agent. The foaming agent should not result in foaming of the foaming gel until it comes in contact with blood. While various foaming agents may be used without departing from the present disclosure, sodium bicarbonate may be utilized in an embodiment of the present disclosure. Upon foaming, presence of the foaming agent in the wound may enable even spreading of pharmaceuticals or other compounds throughout the wound, and the foaming action may apply pressure to the wound that can also resist/stop blood flow. The pharmaceutical hemostat invention may be prepared as a powder or a liquid with no gel added, where the powder can be dusted or poured onto or into the wound and the liquid may be sprayed or squirted or injected onto or into the wound as desired.

In some embodiments of the present disclosure, other compounds may be added at the time that the pharmaceutical-type clotting agent is added, including but not limited to, shock mitigation agents, antibiotics, nano-silver particles at approximately 50-30 nm to kill viruses and bacteria, pain relieving agents such as topical xylocaine, or other pharmaceuticals or additives. Each of these compounds will be described in more detail below.

Upon addition of one or more of these compounds, the resultant foaming gel may be blended until it is stiff in form, and then it may be rolled into one or more thin sheets or it may be formed into a solid shape and dried. In embodiments of the present disclosure, the gel may be then be freeze dried, vacuum dried, or heated in a warm/hot air oven until the thin sheets can be rolled up tightly. The gel may be rolled (i.e., using a sterile non-stick roller) tightly onto a sterile stick or tube or wire until in the shape/thickness desired for insertion into the wound and trimmed to size (see, e.g., FIGURE). The trimmings may then be rolled again to form an additional chemically foaming hemostat if desired in some embodiments of the present disclosure. In another embodiment of the present disclosure, instead of rolling the gel into a sheet, the gel may be poured into a mold, preferably non-stick, and the sterile applicator stick or tube or wire may then be inserted. Regardless what method is utilized for drying, once dry, the chemically foaming hemostat may be placed in a sterile bag capable of gas or radiation or heat sterilization. The sterile bag should be easy to open for fast use in treatment. The construction of the chemically foaming hemostat may be in the form of a non-gel powder or a gel or a non-gel liquid.

As previously described, bleeding may be controlled and/or blood clotting may be accelerated through inclusion of one or more pharmaceuticals or compounds as part of the foaming gel. Clotting agents can include, but are not limited to, 0.2% to 14.5% tranexemic acid with 0.3% to 5% chitosan in a base of 5% to 45% alginic acid calcium salt derivatives or other effective compounds in desired concentrations. While the clotting agents may be pharmaceutical grade, it should be appreciated that other non-pharmaceutical clotting agents may be used in addition to or in place of pharmaceutical clotting agents without departing from the present disclosure.

Infection may be controlled through inclusion of one or more pharmaceuticals and/or compounds, such as through use of nano-sized silver particles. Nano-sized silver particles, usually having a size of under 30 nanometers generally applied at approximately 800 PPM or more, may pass through cell membranes of viruses, bacteria, molds and funguses and kill them by combining with the oxygen in the cell suffocating it and killing the contaminant. Accordingly, these nano-sized silver particles may mitigate infection by killing/controlling/decreasing bacteria, viruses, mold, spores and fungi and other pathogens upon contact. Further, the remaining nano-sized silver particles have a long-term residual anti-infection/protective effect on tissues that remains long after the chemically foaming hemostat foam carrier medium and other antiseptic agents have dried. While embodiments of the present disclosure have disclosed use of nano-sized silver particles to control infection, other pharmaceuticals and/or compounds may be utilized in addition to or in place of nano-sized silver particles to treat infection without departing from the present disclosure.

Shock control may be accomplished by inclusion of one or more pharmaceuticals or compounds, such as 0.025% to 2.5% epinephrine or similar or other products, as part of a foaming gel or powder or liquid. Pain control may be accomplished by inclusion of one or more pharmaceuticals or compounds, such as codeine or other narcotics, liquid lidocaine (2%-10%) or a lidocaine/prilocaine combination (EMLA); however, the type and amount of pain control agent may vary depending on the patient. Sepsis control may be accomplished by inclusion of one or more pharmaceuticals or compounds known to control sepsis, including but not limited to, antiseptics.

A chemically foaming hemostat according to an embodiment of the present disclosure may include a foaming gel or liquid or powder comprised of approximately 0.2% to 14.5% tranexemic acid with approximately 0.3% to 5% chitosan in a base of approximately 5% to 45% alginic acid calcium salt derivatives mixed with approximately 0.025% to 2.5% epinephrine and packaged in a dried gel, liquid gel, solid or powder wound “plug” to keep the pharmaceuticals and compounds in suspension to prevent them from settling out. However, other compositions may be utilized without departing from the present disclosure.

As previously discussed, a chemically foaming hemostat according to an embodiment of the present disclosure may include an insertion mechanism taking the form of a long, sterile stick or tube or wire that may be shaped in a manner to facilitate insertion into a wound, particularly deep wounds, puncture wounds, and internal injuries. A plug formed of the foaming gel and one or more compounds, whether taking the form of a liquid, a gel, or a dried gel, may be affixed to the stick and then pushed into the bottom portion of a wound. The stick may be removed after the contents begin to foam. However, it should be appreciated that leaving the stick in the wound after foaming begins to occur is not believed to have short-term harmful effects and may even be desirable in some embodiments of the present disclosure. It should be appreciated that the plug may be dried into a shape suitable for insertion into a wound, such as sheets that may be rolled together in a tampon-like arrangement. Moving or transportation of the patient may cause bleeding to start again. After application of a foaming hemostat according to an embodiment of the present disclosure, if the wound begins to bleed again, one or more additional foaming hemostats may be applied until bleeding is stopped. This process may be repeated if undesired bleeding begins again.

In some embodiments of the present disclosure, there may be a cover shield tube forming at least part of the insertion mechanism for the chemically foaming hemostat. The stick or tube or wire may hold the chemically foaming hemostat plug in place in the wound as the cover shield tube is withdrawn. The plug portion of the chemically foaming hemostat that contacts the wound generally may assume a rounded shape to facilitate insertion into the wound. However, it may assume other shapes without departing from the present disclosure. It also should be appreciated that the chemically foaming hemostat may be flexible in nature to allow it to follow a circuitous path of the wound during insertion.

In another embodiment of the present disclosure, the chemically foaming hemostat may be a liquid introduced into a wound using a syringe or injection mechanism containing an insertion tube or a similar type applicator that may assist in insertion of the contents of the chemically foaming hemostat into a wound. Upon insertion, the syringe (or a funnel, if used) and insertion tube may be removed. It should be appreciated that the liquid may be an un-dried liquid gel that may be injected into the wound until the wound is filled and begins foaming. The chemically foaming hemostat may include additional tubing, such as an infusion tube, that may allow more or additional pharmaceuticals and/or compounds to be injected into or through the tube into a wound before the insertion mechanism is withdrawn. Additionally or alternatively, introduction of tubing may allow for suction/withdrawal/removal of fluid from the bottom of the wound. It should be appreciated that more than one piece of tubing may be inserted into the wound for addition of pharmaceuticals and/or other compounds, to withdraw fluids, take temperature, pH or other data readings, or heat/cool/circulate fluids or gases associated with the wound without departing from the present disclosure.

The chemically foaming hemostat also may include one or more sensors, detectors, wiring or even fiber optics so that the wound may be monitored and data may be collected. For example, when a gel is rolled around or cast onto a stick to form the chemically foaming hemostat, other wires and/or sensors may be introduced along with the gel so that they can remain in the wound for monitoring (i.e., temperature, pH, pressure), sampling, evacuation, and addition of medications, among other applications. They may then be removed electively at some later time.

In some embodiments of the present disclosure, the chemically foaming hemostat may be formed as a powder (as opposed to a gel) that may be poured, sprinkled or dusted onto a wound. Additionally or alternatively, the chemically foaming hemostat may be supplied as a liquid to be dripped onto or squirted into a wound, and may be used with a syringe having a long dispensing tube. In a further embodiment of the present disclosure, the chemically foaming hemostat may be configured as different sized sheets or patches that may be applied topically to a non-punctured surface wound site or to larger wound sites to address bleeding. Similarly, a liquid or gel forming part of the chemically foaming hemostat may be placed as dots on a wound dressing, a sterile pad, or a bandage to stop blood loss while applying pharmaceuticals and/or other compounds to the wound. The chemically foaming hemostat may have a piercing or cutting device applied to the end of the stick or tube or wire to facilitate cutting through tissues to insert the chemically foaming hemostat into the depth of the wound, or in the case of internal injuries, to facilitate cutting through covering tissues in order for the chemically foaming hemostat to reach the area that is bleeding.

In some embodiments of the present disclosure, a chemically foaming hemostat may contain a radiation or x-ray marker. This radiation marker may allow for easier identification of the wound depth, path and position when the chemically foaming hemostat is introduced into a wound. Further, it may make it easier for medical personnel to find, identify and/or remove the plug or foaming gel portion of the chemically foaming hemostat from the wound at a later time. The marker may be blended into the gel of the chemically foaming hemostat; however, it also may be attached to the chemically foaming hemostat (i.e., may be formed separately from the gel). While the marker has been described as using radiation, it should be appreciated that detection through a marker may be done using x-ray, infrared, ultraviolet radiation, “radioactivity” or other detection mechanisms without departing from the present disclosure. There also may be embodiments of the present disclosure where compounds may be included within the chemically foaming hemostat to dye the foam in a color that may make eventual removal/flushing of the gel/liquid/powder easier.

In some embodiments of the present disclosure, a field dressing may be placed over the wound after the chemically foaming hemostat has stopped the bleeding; however, the field dressing will not be placed inside the wound being treated using the chemically foaming hemostat. For example, when the gel of a chemically foaming hemostat foams, the stick portion may be removed, and a field dressing may be applied. It may be desirable that the field dressing have a non-stick surface so it is less likely to clot onto the wound. Such field dressing may be included in the foaming hemostat packaging. Other wound care accessories may be included in the foaming hemostat packaging including, but not limited to, tweezers, mechanical hemostats, suture equipment, bandages and dressings, “butterfly” bandages, tape, scissors, scalpels, packages or syringes of medicines and pharmaceuticals, medications, writing instruments (such as a laundry market or pen), a tag or label to write on, tourniquets, and other such items as may be desired to be packaged with the chemically foaming hemostat.

While certain pharmaceuticals and compounds have been described herein, it should be appreciated that the number and type of pharmaceuticals or other compounds included as part of the chemically foaming hemostat may be altered depending on the effect or use desired. For example, different pain medications or other therapeutic or non-therapeutic compounds may be included in the chemically foaming hemostat depending on the treatment desired. It also should be appreciated that the quantities may change, and uses/applications other than treatment of bleeding, sepsis, infection, shock and pain and other symptoms may be contemplated though not specifically mentioned herein. It should be appreciated that the dose effectiveness of pharmaceuticals and/or other compounds introduced into a wound may remain constant throughout use. It should further be appreciated that blockage of contaminants from the surface of the wound may continue over an extended period of time, but may be removed through washing/scrubbing, flushing and/or suction techniques in some embodiments of the present disclosure.

In some embodiments of the present disclosure, the chemically foaming hemostat may be applied once to a wound for long-term residual blocking of contaminants; however, there may be other embodiments of the present disclosure where more than one application may be needed/desired. For example, in the case of a large wound, more than one chemically foaming hemostat may be inserted over time or even may be applied to the wound simultaneously. It should be appreciated that the size of a chemically foaming hemostat according to embodiments of the present disclosure may change depending on the type of wound being treated. For example, a chemically foaming hemostat may be provided in a very small diameter size for application into small diameter puncture wounds.

It should be appreciated that the chemically foaming hemostat may be used for treatment of areas other than wounds without departing from the present disclosure. Further, while the chemically foaming hemostat has been described with respect to use on humans, it should be appreciated that it may be used on non-humans without departing from the present disclosure.

It should be appreciated that a chemically foaming hemostat according to embodiments of the present disclosure may be sterilized (such as with gas, radiation or radioactivity) and placed within packaging so that it may have a lengthy shelf life. This may be particularly useful in certain situations, such as on the battlefield or in extreme conditions of moisture, heat, cold, and/or dirt. Even if jostled within the packaging, the chemically foaming hemostat may tolerate some degree of crushing and impact without losing its effectiveness. The packaging should be easy to open so that it can be accessed quickly in an emergency situation to improve a patient's recovery or survival time. It also may be desirable that the packaging holding the chemically foaming hemostat may be lightweight and weatherproof so that field personnel may easily carry dozens of chemically foaming hemostats according to embodiments of the present disclosure. Further, a chemically foaming hemostat according to embodiments of the present disclosure may be easy to use so that a person having little-to-no medical training and expertise may still properly apply the chemically foaming hemostat to a range and sizes and types of wounds. The packaging may include instructions for use in several languages and drawings to instruct its use to the illiterate.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A hemostat comprising:
a foaming composition consisting of:
a gelling agent,
a sterile anhydrous liquid,
a blood-activated foaming agent and
a pharmaceutical-type clotting agent, wherein the foaming composition takes the form of a gel, a sterile anhydrous liquid, or a prescription-grade collagen powder;
an insertion mechanism affixed to the foaming composition,.
wherein the insertion mechanism facilitates introduction of the foaming composition into a wound, and
wherein the foaming composition activatingly contacts blood in at least one of wound tissues, arteries, and veins and abates bleeding upon contact with the blood; and
a foam pressure provided in damaged areas selected from the wound tissues, arteries, and veins
wherein the foam pressure presses the foaming composition into the damaged areas upon contact with blood,
wherein foaming of the hemostat does not occur when the hemostat contacts water, and
wherein the hemostat is anhydrous.

2. The hemostat of claim 1, the foaming composition further consisting of:
one or more compounds selected from the group comprising: clotting agents, shock mitigation agents, antibiotics, nano-silver particles, pain relieving agents, and anti-sepsis agents, wherein upon activation of the foaming composition in the wound, the one or more compounds are introduced into the wound.

3. The hemostat of claim 1, wherein the foaming composition is applied to the insertion mechanism and dried.

4. The hemostat of claim 3 wherein the foaming composition is rolled into thin sheets for application, wherein the thin sheets are affixed to the insertion mechanism.

5. The hemostat of claim 3 wherein the foaming composition is poured into a non-stick mold for application to the insertion mechanism.

6. The hemostat of claim 1, wherein the insertion mechanism is a sterile stick or tube or wire.

7. The hemostat of claim 6, the insertion mechanism further comprising:
a cover shield tube covering the sterile stick or tube or wire, wherein upon insertion of the chemically foaming hemostat into the wound, the sterile stick or tube or wire holds the foaming composition in place and the cover shield tube is withdrawn.

8. The hemostat of claim 1 further comprising:
a radiation or x-ray marker.

9. The hemostat of claim 1 further comprising one or more sensors, detectors or wires to monitor the wound.

10. The hemostat of claim 1 further comprising one or more tubes to evacuate and introduce liquid into the wound.

11. A gel for use in a chemically foaming hemostat, the gel consisting of:
a first mixture formed from mixing a gelling agent with a sterile anhydrous liquid;
a blood-activated foaming agent and a pharmaceutical-type clotting agent added to the first mixture and blended to form the gel of the chemically foaming hemostat, wherein the gel activatingly contacts blood in at least one of wound tissues, arteries, and veins and abates bleeding in the wound tissues, arteries, and veins upon contact with the blood; and
a foam pressure provided in damaged areas selected from the wound tissues, arteries, and veins
wherein the foam pressure presses the gel into the damaged areas upon contact with blood,
wherein foaming of the hemostat does not occur when the hemostat contacts water, and
wherein the hemostat is anhydrous.

12. The gel of claim 11, the gel further consisting of:
one or more additional compounds selected from the group comprising: shock mitigation agents, antibiotics, nano-silver particles, pain relieving agents, and anti-sepsis agents.

13. The gel of claim 11 wherein the gel is rolled into one or more thin sheets and affixed to a sterile stick or wire that is used to introduce the gel into the wound, wherein upon application, the gel containing the sterile stick or wire is dried.

14. The gel of claim 11 wherein the gel is formed into a solid shape and applied to a sterile stick or wire that is used to introduce the gel into the wound, wherein upon application, the gel containing the sterile stick or wire is dried.

15. The gel of claim 11 wherein the gel is poured into a non-stick mold, a sterile stick or wire is inserted into the gel, and the gel containing the sterile stick or wire is dried.

16. The gel of claim 11 wherein the gel remains in liquid form for introduction into the wound through the insertion mechanism.

17. The gel of claim 11 wherein the gel is placed into powder form for introduction into the wound.

18. The gel of claim 11 wherein the gel is formed as sheets to be applied topically to the wound.

19. A method for using a chemically foaming hemostat, the method comprising:
introducing the chemically foaming hemostat formed of a foaming agent affixed to a sterile stick or wire into a wound where blood is present; and
removing the sterile stick or wire from the wound when the foaming agent has been introduced into the wound, wherein upon contact with the blood, the foaming agent activates to foam in the wound and bleeding associated with the wound stops.

20. The method of claim 19 wherein the foaming agent is impregnated with one or more compounds selected from the group comprising: clotting agents, shock mitigation agents, antibiotics, nano-silver particles, pain relieving agents, and anti-sepsis agents, wherein upon activation of the chemically foaming hemostat in the wound, the one or more compounds are introduced into the wound.
